(19) United States
(12) Patent Application Publication
(10) Pub. No.: US 2024/0285575 A1
(43) Pub. Date: Aug. 29, 2024

(54) NANOPARTICLES WITH ANTI-INFLAMMATORY PROPERTIES AND ENHANCED PENETRATION ACROSS THE BIOLOGICAL BARRIERS TO DELIVER ENCAPSULATED DRUGS

(71) Applicant: The Board of Trustees of The University of Alabama, Tuscaloosa, AL (US)

(72) Inventors: Ravikumar Majeti, Tuscaloosa, AL (US); Meenakshi Arora, Tuscaloosa, AL (US); Ingrid Marie Heyns, Tuscaloosa, AL (US); Raghu Ganugula, Tuscaloosa, AL (US)

(21) Appl. No.: 18/397,327

(22) Filed: Dec. 27, 2023

Related U.S. Application Data
(60) Provisional application No. 63/448,405, filed on Feb. 27, 2023.

(51) Int. Cl. A61K 31/353; A61K 9/51; A61K 47/59; A61K 47/60; A61P 5/50; A61P 29/00

(52) U.S. Cl. CPC A61K 31/353; A61K 9/5153; A61K 47/593; A61K 47/60; A61P 5/50; A61P 29/00

(57) ABSTRACT

Disclosed herein are compositions of nanoparticles with naringenin as a folate receptor (FR)-targeting ligand and methods of making and administering the nanoparticles with naringenin (NAR) ligands for drug delivery applications. The disclosed compositions include NAR-functional polyester-based nanoparticles encapsulating insulin (i.e. insulin-laden NAR-functional nanoparticles), which showed loading capacities of 10% and encapsulation efficiency of >70%. The insulin-laden NAR-functional nanoparticles are shown in examples to have a 3-fold higher bioavailability of insulin compared to unfunctionalized nanoparticles, suggesting the role of receptor-mediated transport across the intestinal barriers. Methods of making NAR-functional nanoparticles are also disclosed herein, and methods of administration for the peroral delivery of encapsulated drugs.

### CROSS-REFERENCE TO RELATED APPLICATIONS

This application claims the benefit of priority to U.S. Provisional Application No. 63/448,405, filed Feb. 27, 2023, which is incorporated herein by reference in its entirety.

### STATEMENT OF GOVERNMENT SUPPORT

This invention was made in parts with government support under Grant nos. R01DK125372 and R01DK128597 awarded by the National Institutes of Health. The government has certain rights in the invention.

### TECHNICAL FIELD

The disclosure relates to compositions and methods of making decorated nanoparticles for improved bioavailability of drugs.

### BACKGROUND

Improving the bioavailability of drugs, particularly for enteral delivery has various challenges due to crossing the biological barriers and enduring varying pH conditions in the stomach and gastrointestinal tract. Nanoparticles assist crossing the biological barriers and protecting the encapsulated drugs against harsh environments. Nanoparticles display advantageous physiochemical properties and can impart various functionalities, mainly directed towards targeting ligands. Numerous small molecule ligands have since been identified for receptor-mediated drug delivery, with the continual objective to pursue ligands with high affinity towards biological receptors, e.g. transferrin receptors (TfR) and folate receptors (FR).

The oral delivery of proteins, specifically the peroral delivery of insulin, has become an extensively studied area due to the advantages of patient compliance over subcutaneous injection. Insulin encounters its primary obstacles in the gastrointestinal tract due to degradation by proteolytic enzymes and inadequate transport across the intestinal epithelium. Improving the bioavailability of these drugs/biologics, particularly for enteral delivery, has various challenges due to crossing the biological barriers and enduring low-pH conditions in the gastrointestinal tract. In recent years, nanoparticles have been employed due to their advantageous physiochemical properties and their ability to impart a plethora of functionalities, mainly directed toward receptor-specific targeting ligands. Folate, neonatal Fc, and transferrin receptors are among the few transcytosis targeted receptors used in oral drug delivery. Several strategies for receptor targeting ligands have been employed, including peptides, aptamers, antibodies, and small molecules. However, small molecule ligands remain of high interest due to their simplicity and scalability,¹³ in contrast with other targeting ligands suffering from variation in activity upon slight structural modifications, unscalable processes,¹⁴ configuration-dependent degradation,¹⁵ and high cost and toxicity. Numerous small molecule ligands have since been identified for receptor-mediated drug delivery, with the continual objective to pursue ligands with high specificity toward receptors.

Naringenin, a flavanone that forms part of the class of citrus polyphenols, provides various biological and pharmacological properties, including anti-inflammatory, anticancerous, neuroprotective, and antioxidative effects. Naringenin is poorly bioavailable upon oral administration, which is partly contributed by efflux transporters P-glycoprotein (P-gp), multidrug resistance-related protein 2 (Mrp2), and breast cancer resistance protein (Bcrp1). Attempts were made to improve oral bioavailability of NAR by encapsulating into nanoparticles. Nanoparticles have been reported to improve drug delivery by overcoming efflux-mediated poor bioavailability.

### SUMMARY

Disclosed herein are compositions of nanoparticles with naringenin as a folate receptor (FR)-targeting ligand and methods of making and administering the nanoparticles with naringenin (NAR) ligands for drug delivery applications. The disclosed compositions include NAR-functional polyester-based nanoparticles encapsulating insulin (i.e. insulin-laden NAR-functional nanoparticles), which showed loading capacities of 10% and encapsulation efficiency of >70%. The insulin-laden NAR-functional nanoparticles are shown in examples to have a 3-fold higher bioavailability of insulin compared to unfunctionalized nanoparticles, suggesting the role of receptor-mediated transport across the intestinal barriers. Methods of making NAR-functional nanoparticles are also disclosed herein, and methods of administration for the peroral delivery of encapsulated drugs.

Additionally disclosed are examples of synthesis of NAR-functional nanoparticles and the interaction of the disclosed nanoparticles with folate receptor targets by NAR-FR binding. Additional examples show in vitro validation for receptor mediated transport.

In some aspects, the techniques described herein relate to a composition including a nanoparticle, wherein the nanoparticle includes a polymer or copolymer conjugated to a naringenin ligand.

In some aspects, the techniques described herein relate to a composition, further including a bioactive drug encapsulated by the nanoparticle.

In some aspects, the techniques described herein relate to a composition, wherein the bioactive drug has at least one of limited aqueous solubility or limited tissue penetration.

In some aspects, the techniques described herein relate to a composition, wherein the drug is naringenin, insulin, GLP-1 agonist, curcumin, urolithin A, cyclosporine A, or combinations thereof.

In some aspects, the techniques described herein relate to a composition, wherein the polymer or copolymer includes polyethylene glycol, polylactide, poly(lactide-co-glycolide), polycaprolactone, polyglycolide, polyhydroxyalkanoates, polyanhydrides, polyurethanes, polyphosphazenes or combinations thereof.

In some aspects, the techniques described herein relate to a composition, wherein the polymer or copolymer is a block copolymer including a polylactide block and a polyethylene glycol block.

In some aspects, the techniques described herein relate to a composition, wherein the block copolymer is a triblock, multiblock, or starblock copolymer.

In some aspects, the techniques described herein relate to a composition, wherein the naringenin ligand is linked to carboxylic end groups of the polymer or copolymer by a linker.

In some aspects, the techniques described herein relate to a composition, wherein the linker includes an amino acid or bi- and multi-functional diamines, wherein the amino acid, bi- and multi-functional diamines include alkyl chains of lengths from C1 to C6.

In some aspects, the techniques described herein relate to a composition, wherein the linker includes polyethylene glycol (PEG) chains, wherein the PEG chains are less than 5 kDa.

In some aspects, the techniques described herein relate to a composition, wherein the amino acid includes β-boc alanine, γ-(boc-amino) butyric acid or 6-(boc-amino) caproic acid.

In some aspects, the techniques described herein relate to a composition, wherein a ratio of polymer or copolymer to naringenin ligand is varied from 4 to 12 naringenin ligands to every polymer or copolymer.

In some aspects, the techniques described herein relate to a composition, wherein the nanoparticles are configured to selectively target folate receptors.

In some aspects, the techniques described herein relate to a composition further including one or more detectable compounds chosen from fluorophoric, radio-labeled, and inorganic compounds, wherein the detectable compounds are encapsulated or conjugated to the nanoparticles.

In some aspects, the techniques described herein relate to a method of producing a composition, the method including: synthesizing a polymer or copolymer conjugated with naringenin; and preparing the nanoparticles by entrapping a desired bioactive drug within the nanoparticles.

In some aspects, the techniques described herein relate to a composition, further including a suspension of freeze-dried nanoparticles in water or milk, thereby forming uniform colloidal system.

In some aspects, the techniques described herein relate to administration of the composition to a subject.

In some aspects, the techniques described herein relate to administration of the composition, wherein the administering is carried out orally or perorally.

In some aspects, the techniques described herein relate to administration of the composition, wherein the administering is by nose, skin, or injection.

In some aspects, the techniques described herein relate to administration of the composition, wherein the composition is administered in response to short-term or chronic inflammation of a subject.

### BRIEF DESCRIPTION OF THE DRAWINGS

FIG. 1A shows a synthesis route of polymer (P2s-NAR), and FIG. 1B shows ¹H NMR spectra of 2 and P2s-NAR in DMSO-d₆.

FIGS. 1C-1E show mass spectrum of compound 1 (FIG. 1C); ¹H NMR spectrum of compound 1 (FIG. 1D) in DMSO-d₆; and ¹³C NMR spectrum of compound 1 (FIG. 1E) in DMSO-d₆.

FIG. 1F-1H show: mass spectrum of compound 2 (FIG. 1F); ¹H NMR spectrum of compound 2 (FIG. 1G) in DMSO-d₆, and ¹³C NMR spectrum of compound 2 (FIG. 1H) in DMSO-d₆.

FIG. 2A. shows GPC chromatogram of a prepolymer, P2s and P2s-NAR.

FIG. 2B shows ¹H NMR spectrum of the prepolymer in CDCl₃.

FIGS. 3A-3B show UV-Vis spectra of P2s, NAR and P2s-NAR (FIG. 3A) and FTIR spectra of P2s and P2s-NAR (FIG. 3B).

FIGS. 4A-4B show SEM micrographs of insulin-laden P2Ns (FIG. 4A) and insulin-laden P2Ns-NAR (FIG. 4B).

FIGS. 5A-5E show naringenin-laden P2s/P2s-NAR nanoparticle formulations and characterizations including SEM micrographs of P2Ns (FIG. 5A), NAR-laden P2Ns (FIG. 5B), P2Ns-NAR (FIG. 5C), NAR-laden P2Ns-NAR (FIG. 5D), and tabulated particle sizes (DLS), zeta potentials and entrapment efficiencies of respective nano-formulations (FIG. 5E).

FIG. 6 shows representative scanning electron micrographs of NAR-laden PLGA-NAR.

FIGS. 7A-7B show the DLS intensity plots of insulin-laden nano-formulations (FIG. 7A) and particle size distribution of insulin-laden nano-formulations via SEM (FIG. 7B), which includes 50 nanoparticles for particle size distribution from each SEM micrograph.

FIGS. 8A-8F show: in FIG. 8A, confocal micrographs of cellular uptake of C6-laden P2Ns and P2Ns-NAR in FHs74 cell lines; in FIG. 8B, co-labeling of C6-laden P2Ns and P2Ns-NAR (green), nuclei (Hoechst staining, blue), and FR (red) for colocalization studies in FHs74 cell lines via CLM; in FIG. 8C, percentage cellular uptake of C6-laden P2Ns and P2Ns-NAR in FHs74 cell lines, determined via flow analysis; in FIG. 8D Colocalization coefficient of C6 with FR, determined respectively for P2Ns and P2Ns-NAR from confocal colocalization studies; in FIG. 8E, Mander's coefficient of C6 with FR, determined respectively for P2Ns and P2Ns-NAR from confocal micrographs; and in FIG. 8F, Pearson's correlation coefficient of C6 with FR, determined respectively for P2Ns and P2Ns-NAR from confocal micrographs.

FIGS. 9A-9C show: in FIG. 9A, percentage cellular uptake efficiency obtained by flow cytometry density plots of cellular uptake of P2Ns, P2Ns-NAR (dispersed in water) in FHs74 cells at 37° C. (50 μg/mL); in FIG. 9B, percentage cellular uptake efficiency obtained by flow cytometry density plots of cellular uptake of P2Ns, P2Ns-NAR (dispersed in formula milk) in FHs74 cells at 37° C. (50 μg/mL); in FIG. 9C, colocalization co-efficient of C6 with FR, determined respectively for P2Ns and P2Ns-NAR from confocal colocalization studies.

FIGS. 10A-10C show folate receptor blocking studies in the FHs74 cell lines using varying concentration of antifolate antibody (AB) (FIG. 10A), varying concentration of folic acid (FIG. 10B), and varying concentration of methyltetrahydrofolic acid (MTHFA) (FIG. 10C).

FIG. 11 shows insulin plasma concentration profiles for P2Ns and P2Ns-NAR, in vivo, over 96 hours.

FIG. 12A-12B show synthesis routes of PLGA-NAR (FIG. 12A), and ¹H NMR spectra of PLGA-NAR in DMSO-d₆ (FIG. 12B).

FIGS. 13A-13B show: in FIG. 13A, TLR4 expression and cellular death of FHs74 cells upon exposure to 50 μg/mL LPS for 1 hour; and in FIG. 13B, after 6 hours exposure upon treatment with unfunctionalized nano-formulations versus NAR-decorated nano-formulations. Naringenin, void and naringenin-laden were used in treatment groups (100 μM).

FIGS. 14A-14B show control datasets of human small intestine cell lines treated with NAR and LPS respectively. FIG. 14A shows TLR4 expression and cell viability of FHs74 cells upon 1 hour exposure to varying concentrations of free NAR, and FIG. 14B shows FR expression and cell viability of FHs74 cells upon 1 hour exposure to varying concentrations of LPS.

FIGS. 15A-15J show: in FIG. 15A, MFI determined by flow cytometry to evaluate the cellular uptake in HK2 cells; in FIG. 15B efficacy study of TLR4, FR expression and cell death of cisplatin treated (20 μM) HK2 cells as a function of treatment groups, determined via FACS; in FIG. 15C, NFkB and IL1β expression of HK2 cells upon exposure to varying concentrations of cisplatin for 6 hours; and in FIGS. 15D-15J, inflammation assays. A: Control; B: CIS Control; C: CIS+NAR; D: CIS+P2Ns (void nanoparticles); E: CIS+P2Ns-(NAR) (NAR-laden nanoparticles); F: CIS+P2Ns-NAR [void nanoparticles decorated with NAR]; G: CIS+P2Ns-NAR-(NAR) [NAR-laden nanoparticles decorate with NAR].

FIG. 16 shows TLR4 expression and cellular death of FHs74 cells upon exposure to 30 mM glucose (high glucose) for 3 hours upon treatment with unfunctionalized nano-formulations versus NAR-decorated nano-formulations. Naringenin, void and naringenin-laden were used in treatment groups.

FIG. 17 shows in vivo efficacy studies of NAR-decorated nano-formulations. [NAR, 20 mg/kg daily dose; P2Ns (NAR) 20 mg/kg (NAR equivalent encapsulated in the particles) 3 doses/week for 4 weeks; P2Ns-NAR (10 mg/kg (NAR equivalent, conjugated to the polymer) 3 doses/week for 4 weeks; P2Ns-NAR (NAR) (10 mg/kg (NAR equivalent encapsulated in the particles) 3 doses/week for 4 weeks; PLGA-NAR (NAR) (10 mg/kg (NAR equivalent encapsulated in the particles) 3 doses/week for 4 weeks.

FIGS. 18A-18D show Efficacy of oral UA-laden nano-formulations as a function of unfunctionalized nano-formulations formulations (20 mg/kg body weight, UA equivalent) versus NAR-decorated nano-formulations (10 mg/kg body weight, UA equivalent) in cisplatin-induced acute kidney mice models.

FIG. 19 shows UA concentration in oral UA-laden nano-formulations (20 mg/kg body weight, UA equivalent) as a function of unfunctionalized nano-formulations versus NAR-decorated nano-formulations (10 mg/kg body weight, UA equivalent) in cisplatin-induced acute kidney mice models.

### DETAILED SPECIFICATION

Some example devices and methods of implantation are described in the following section.

In one embodiment, a composition is disclosed, the composition including a nanoparticle, wherein the nanoparticle comprises a polymer or copolymer conjugated to a naringenin ligand. The naringenin ligand is used, for its experimentally verified selectively, to target folate receptors.

In some embodiments, the naringenin-conjugated polymer or copolymer nanoparticle further includes a bioactive drug encapsulated by the nanoparticle. The bioactive drug is described by at least one of limited aqueous solubility or limited tissue penetration. In some examples, the bioactive drug is naringenin, insulin, GLP-1 agonist, curcumin, urolithin A, cyclosporine A, or combinations thereof.

In some embodiments, the polymer or copolymer comprises polyethylene glycol, polylactide, poly(lactide-co-glycolide), polycaprolactone, polyglycolide, polyhydroxyalkanoates, polyanhydrides, polyurethanes, polyphosphazenes or combinations thereof. In one example, the copolymer is a block copolymer comprising a polylactide block and a polyethylene glycol block, wherein the block copolymer is a triblock, multiblock, or starblock copolymer.

In some embodiments, the naringenin ligand is linked to carboxylic end groups of the polymer or copolymer by a linker. In some examples, the linker comprises an amino acid or bi- and multi-functional diamines, wherein the amino acid, bi- and multi-functional diamines comprise alkyl chains of lengths from one to six carbons. In one example, the linker comprises polyethylene glycol (PEG) chains. For example, the PEG chains are less than 1 kDa, less than 2 kDa, less than 3 kDa, less than 4 kDa, less than 5 kDa, less than 6 kDa, less than 7 kDa, less than 8 kDa, less than 9 kDa, or less than 10 kDa.

In other examples, the amino acid comprises β-boc alanine, γ-(boc-amino) butyric acid or 6-(boc-amino) caproic acid.

In some embodiments, the naringenin-conjugated polymer or copolymer nanoparticle includes a ratio of polymer or copolymer to naringenin ligand that is varied from 4 to 12 naringenin ligands to every polymer or copolymer.

In some embodiments, the naringenin-conjugated polymer or copolymer nanoparticle further includes one or more detectable compounds chosen from fluorophoric, radio-labeled, and inorganic compounds. In some examples, the detectable compounds are encapsulated or conjugated to the nanoparticles.

In some embodiments, a method of producing the composition including the naringenin-conjugated polymer or copolymer nanoparticle is disclosed. The method includes synthesizing a polymer or copolymer conjugated with naringenin. The method may further include preparing the nanoparticles by entrapping a desired bioactive drug within the nanoparticle.

In some embodiments, a uniform colloidal system is formed from the composition. The uniform colloidal system includes freeze-dried nanoparticles of the composition with or without encapsulated bioactive drug, suspended in water or milk.

In some embodiments, a method of administration is disclosed. The method includes administering, to a subject, the uniform colloidal system of the disclosed composition with the bioactive drug. The uniform colloidal system may be a suspension of freeze-dried nanoparticles of the disclosed composition in water or milk.

In some examples, the method of administration is carried out orally or perorally. In other examples, the method of administration is carried out by nose, skin, or injection.

In some examples, the method of administration is carried out in response to short-term or chronic inflammation of a subject.

In view of the described processes and compositions, hereinbelow are described certain more particularly described aspects of the inventions. These particularly recited aspects should not, however, be interpreted to have any limiting effect on any different claims containing different or more general teachings described herein, or that the “particular” aspects are somehow limited in some way other than the inherent meanings of the language and formulas literally used therein.

The present invention may be understood more readily by reference to the following detailed description of various aspects of the invention and the examples included therein and to the Figures and their previous and following description.

### Examples

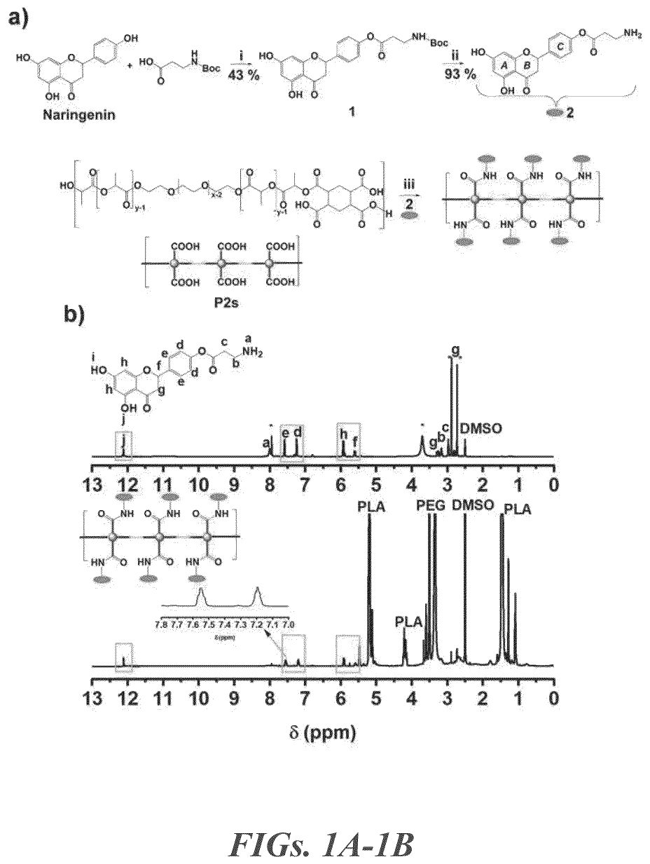

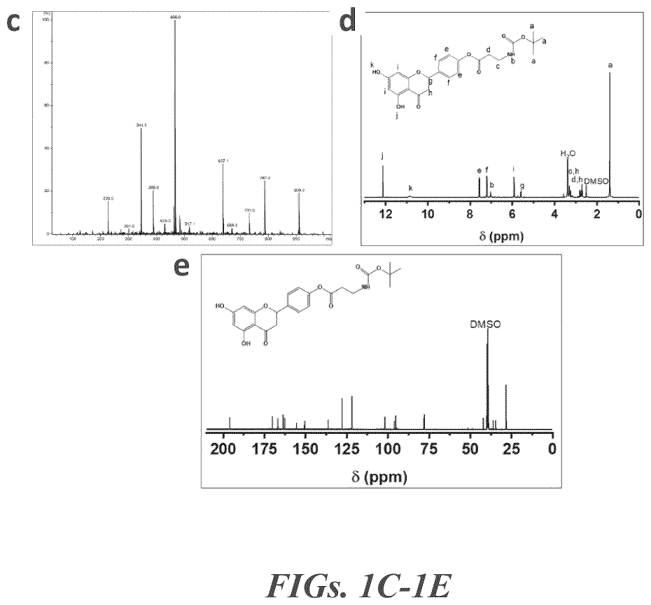

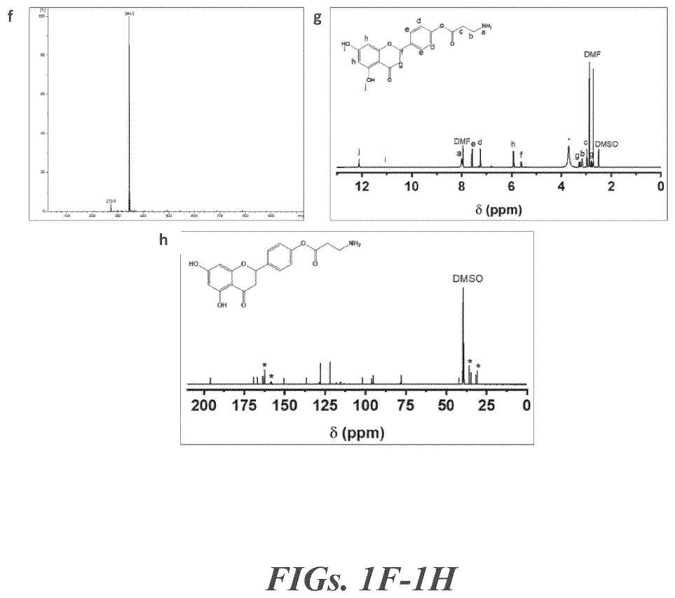

Example: Synthesis and Characterization of Naringenin-Conjugated Precision Polyesters (P2s-NAR).³⁷ A naturally occurring amino acid, β-boc alanine, was introduced as a linker to the ligand NAR via a Steglich esterification, yielding the monosubstituted analogue 1, presented in FIG. 1A (¹H,¹³C NMR and MS are shown in FIGS. 1C-1E). The moderate yield of this reaction was ascribed to the simultaneous formation of the disubstituted β-boc alanine NAR analogue at rings A and C.²⁰ Nonetheless, subsequent removal of the N-boc group gave the primary amine NAR analogue 2, in good yields (FIGS. 1A and FIGS. 1F-1H). It is contemplated that optimizing the ligand density plays an important role in governing the nanoparticle-receptor interaction and improved cellular.^{11,21}

The synthetic route, as shown in FIG. 1A recites i) EDC·HCl, DMAP, dimethylformamide, 0° C. to room temperature; ii) trifluoroacetic acid, methanol, dichloromethane, 0° C.; iii) EDC·HCl, DIEA, dimethylformamide, dichloromethane, 0° C. to room temperature.

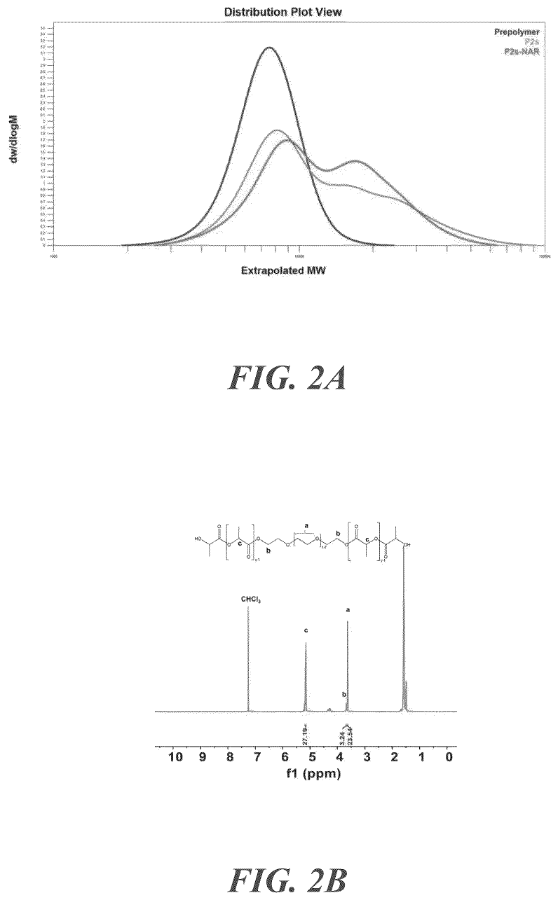

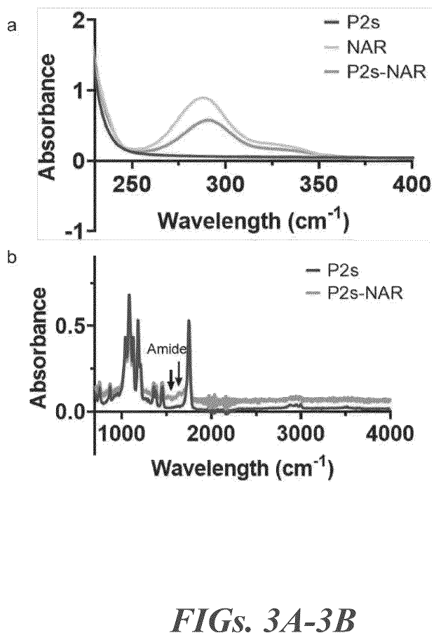

Next, the NAR-analogue 2 was conjugated to a precision polyester polymer (P2s).¹¹ The high functionality of P2s allows for increased ligand-receptor stoichiometry. P2s consists of multiple polylactide-poly(ethylene glycol)-polylactide (PLA-PEG-PLA or prepolymer) triblocks linked via a cyclohexanetetracarboxylic dianhydride (HCDA) spacer, imparting pendant carboxylic acid moieties periodically along the polymeric backbone (FIGS. 1A and 1). The number-average molecular weight (Mₙ) of P2s was determined to be 10000 g/mol, D=1.46, and after NAR conjugation, Mₙ=10800 g/mol, D=1.35 via gel permeation chromatography (GPC). The amine of the NAR-alanine, 2, was coupled to the carboxylic acid functionalities of P2s via 1-ethyl-3-(3-(dimethylamino)propyl)carbodiimide (EDC) coupling. FIG. 1B represents the ¹H NMR spectra of 2 and conjugation of 2 to P2s, P2s-NAR. The broadening of the aromatic peaks in ¹H NMR was attributed to the NAR moiety, which suggests the successful incorporation of 2 onto the polymeric chain. Successful conjugation of 2 to P2s was confirmed by diffusion-ordered spectroscopy (DOSY) NMR, such that NAR moieties' resonances were aligned with the corresponding PLA and PEG resonances attributed by P2s. P2Ns and P2Ns-NAR were subjected to gel permeation chromatography, and Mₙ of approximately 10000 g/mol was observed (FIGS. 2A and 2B). UV-vis spectra of P2s-NAR revealed an absorbance at approximately 280 nm, corresponding to NAR, and FTIR spectra of P2s-NAR confirmed amide bond formation (FIGS. 3A and 3B), indicative of successful EDC coupling of 2 to P2s.

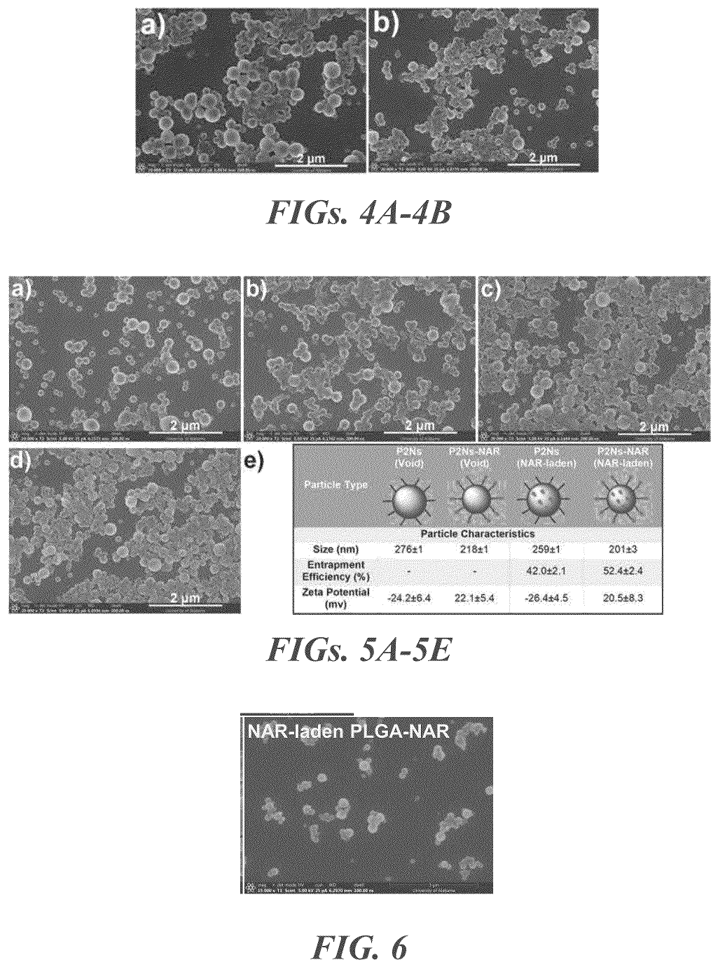

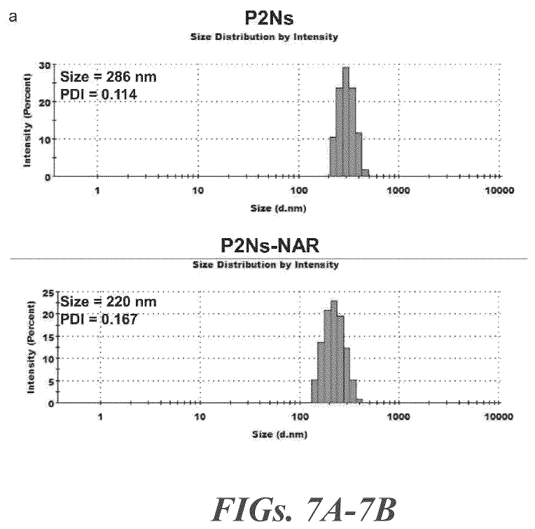

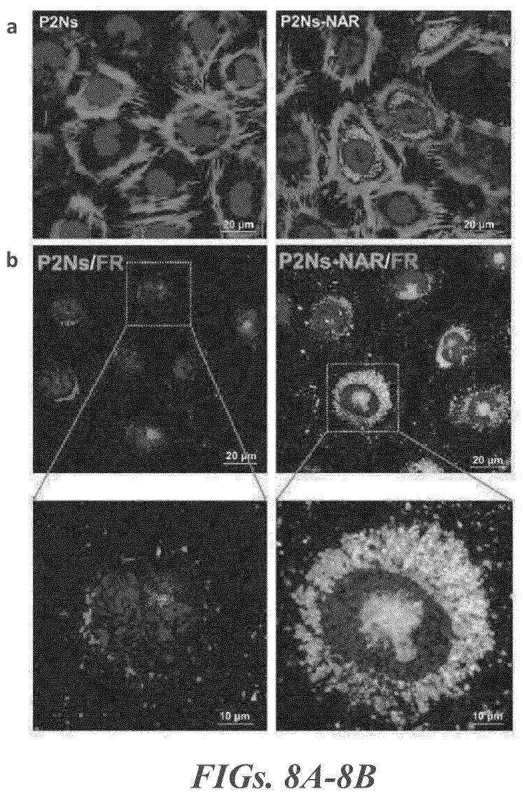

To validate the hypothesis that the uptake was FR mediated, fluorophoric coumarin-6-laden nanoparticles (abbreviated as C6-laden P2Ns or P2Ns-NAR) were prepared via oil-in-water (O/W) emulsification and were used in flow cytometry (FC) and confocal laser scanning microscopy (CLSM) studies. Subsequently, insulin-laden P2Ns and P2Ns-NAR were prepared via a water-oil-water (W/O/W) emulsification process, resulting in 77% and 75% entrapment efficiencies, respectively (at 10% insulin loading), as shown in Table 2. The insulin entrapment efficiencies were slightly higher than other insulin-laden polyester nanoparticles reported in the literature.²²⁻²⁵ SEM images of insulin-laden P2Ns (FIG. 4A) and insulin-laden P2Ns-NAR (FIG. 4B) nanoparticles show spherical, smooth nanoparticles, of various particle sizes in relatively moderate agreement with the hydrodynamic diameters observed from DLS, ranging from 220 to 290 nm (per Table 2 and FIGS. 7A and 7B), which is an appropriate size range for oral absorption.^{11,22}

In addition, all zeta potentials were above ±10 mV, indicative of relatively stable emulsions with relatively less repulsion between nanoparticles, in turn avoiding undesirable particle aggregation (Table 1).²⁶

TABLE 1
Tabulated Particle Sizes (from DLS), Zeta Potentials,
and Entrapment Efficiencies of Respective Nano-formulations,
and Corresponding Standard Deviations

| | | Entrapment | Zeta |
|---|---|---|---|
| | Size ^{a} | efficiency ^{b} | potential ^{a} |
| Particle characteristics | (nm) | (%) | (mV) |
| P2Ns (INS-laded) | 289 ± 12 | 77.0± | −16.3 ± 0.1 |
| P2Ns-NAR (INS-laden) | 220 ± 22 | 75.0 ± 12.1 | 13.9 ± 1.4 |

^{a} Determined from DLS.
^{b} Determined from HPLC.

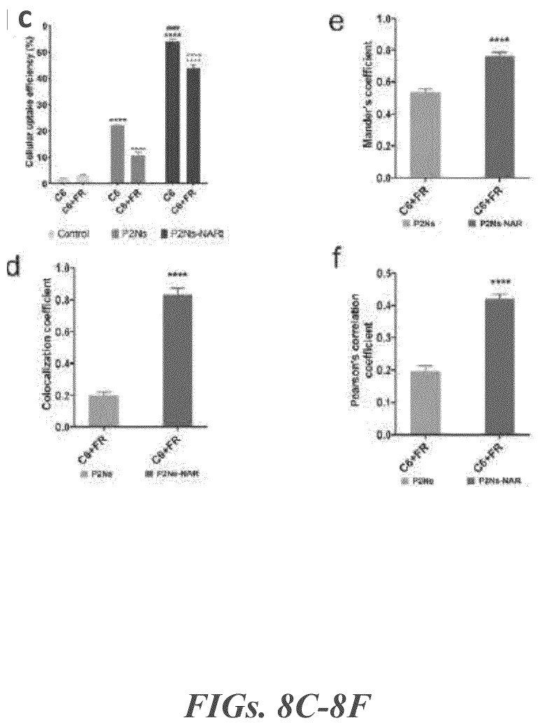

Cellular Uptake. To demonstrate the uptake of the P2Ns-NAR via the enteral route, FHs74 (human fetal small intestinal epithelial cell line) cells were used to mimic the uptake in the gastrointestinal tract in vitro. To gain insight into the translocation of the nanoparticles, CLSM analysis of colocalization demonstrated a significant 4-fold increased colocalization of the P2Ns-NAR and FR in comparison to P2Ns and FR, suggesting strong interactions between FR and NAR (FIGS. 8C-8F). The qualitative uptake of fluorophoric nanoparticles C6-laden P2Ns and P2Ns-NAR in FHs 74 cells are shown in FIGS. 8A-8B after 1 hour of incubation. A higher population of internalized nanoparticles was observed for the NAR-decorated particles, ascribed to the proposed receptor-mediated endocytosis via CLSM. The FR-mediated uptake of the nanoparticles was evaluated by studying the colocalization of the fluorescent nanoparticles with immune-labeled FR via CLSM (FIG. 8C). C6-P2Ns indicated endocytosis likely via nonspecific interactions; however, C6-laden P2Ns-NAR indicated a significantly higher percentage FR colocalization (FIG. 8D). Colocalization, Mander's, and Pearson's coefficients were calculated from cytofluorograms obtained from CLSM. All coefficients for P2Ns-NAR were significantly higher than for P2Ns (FIGS. 8D-8F), further supporting the P2Ns-NAR FR mediated transport.

Furthermore, the cellular uptake efficiency of C6-laden P2Ns and P2Ns-NAR was monitored upon dispersion of nanoparticles in commercially available formula milk, proposed to assist in the dosing of the nanoparticles. The bioavailability of orally administered drugs can be influenced by various factors including the presence of food in the gastrointestinal tract. The composition of the food can affect the rate and extent of drug absorption as well as its distribution and elimination from the body. P2Ns-NAR indicated excellent cellular uptake compared with P2Ns in formula milk. The ingredients present in formula milk could serve as an explanation for the improved uptake, as dietary factors frequently influence the bioavailability of polyphenols.^{27,28}

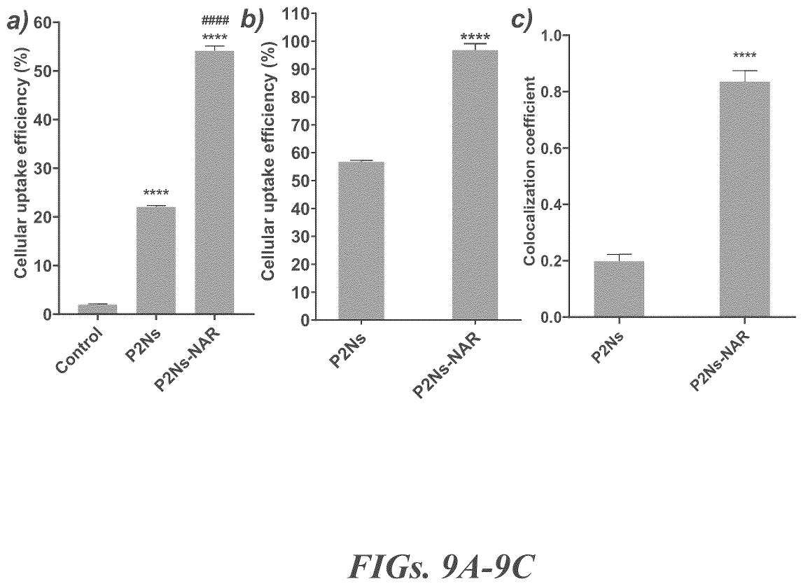

Additional cellular uptake experiments are shown in FIGS. 9A-9C. In FIG. 9A, percentage cellular uptake efficiency obtained by flow cytometry density plots of cellular uptake of P2Ns, P2Ns-NAR (dispersed in water) in FHs74 cells at 37° C. (50 μg/mL); in FIG. 9B, percentage cellular uptake efficiency obtained by flow cytometry density plots of cellular uptake of P2Ns, P2Ns-NAR (dispersed in formula milk) in FHs74 cells at 37° C. (50 μg/mL); in FIG. 9C, colocalization co-efficient of C6 with FR, determined respectively for P2Ns and P2Ns-NAR from confocal colocalization studies.

However, the finding that P2Ns-NAR exhibited improved cellular uptake efficiency in formula milk could have important implications for the pharmacokinetic profile of the nanoparticles in vivo. In the case of P2Ns-NAR, the improved uptake in formula milk suggests that the nanoparticles may exhibit enhanced bioavailability when administered orally in the presence of food. This could potentially translate to higher plasma concentrations of insulin and a longer duration of action, as observed in the pharmacokinetic study.

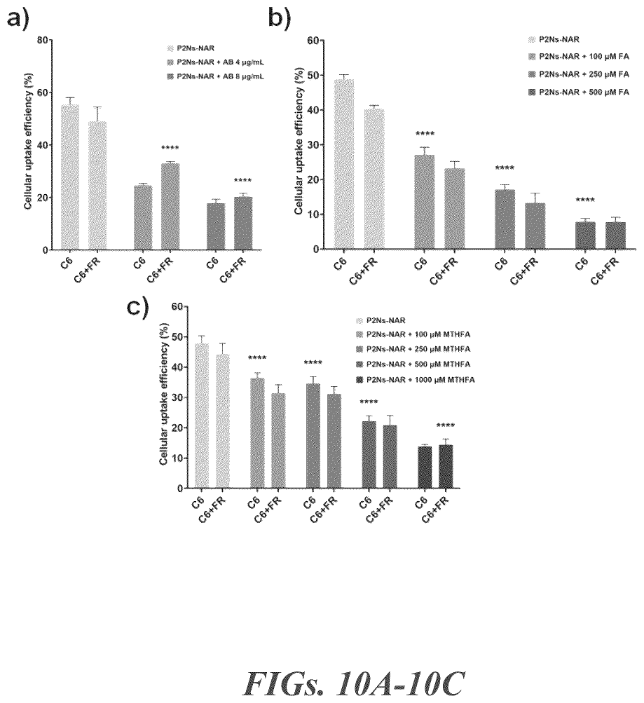

FR Specificity to NAR. The FR-mediated transport of P2Ns-NAR was confirmed by performing blocking studies. Folic acid (FA) strongly binds to FR, with the FA frequently being employed as a targeting ligand on nanoparticles for receptor-mediated drug delivery.^{29,30} On the other hand, FA in larger doses is also associated with adverse side effects including toxicity to humans.^{16,31,32} The use of NAR as an FR-specific ligand could potentially overcome this limitation and provide a safer alternative for receptor-mediated drug delivery. Selective blockade of FR on FHs74 cells was performed to evaluate the FR-mediated uptake of P2Ns-NAR. Cells were saturated with varying concentrations of blocking agents: antifolate antibody (AB), FA, and MTHFA. Cells were allowed to saturate with the respective blocking agents for 1 hour, which was informed by a previous study indicating FA saturates FHs74 cells in less than 1 hour with FR expression remaining constant for a further 6 hours.³³ The cellular uptake efficiency of P2Ns-NAR decreased incrementally with increasing concentrations of the blocking agent, indicating that the uptake was FR-mediated. The response to addition of increasing concentrations AB are shown in FIG. 10A. Higher concentrations of MTHFA (FIG. 10C) were required to achieve blocking levels similar to those achieved with FA (FIG. 10B). This was ascribed to the naturally occurring form of FA, MTHFA, exhibiting superior bioavailability, specifically in the gastrointestinal tract, independent of the pH of the environment.³⁴ Thus, requiring higher concentrations of MTHFA were required to achieve equivalent saturation levels of the FR compared to FA. These results confirm the specificity of NAR as a ligand for FR-mediated drug delivery.

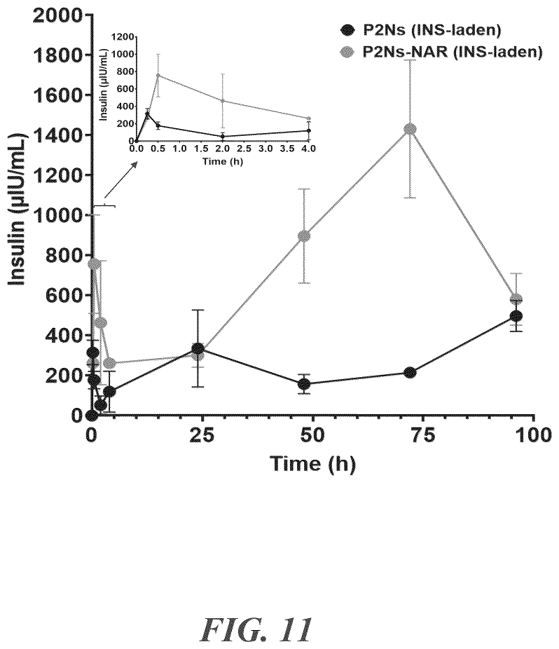

In vivo study. To further demonstrate the FR-mediated uptake of P2Ns-NAR, a single-dose pharmacokinetic study was conducted in male Sprague-Dawley rats (n=4). Rats were orally dosed with insulin-laden P2Ns and P2Ns-NAR (40 IU/kg insulin equivalent). Insulin plasma concentrations were measured over a 96-hour period (FIG. 11). The pharmacokinetic parameters are listed in Table 2. NAR-functional nanoparticles demonstrated a 3-fold increased bioavailability in systemic circulation in comparison to unfunctional P2Ns (Table 2), based on the area under curve (AUC). This suggested that the addition of NAR functionalization to the nanoparticles enhanced their uptake by the cells expressing the FR receptor in the gastrointestinal tract, leading to an increased systemic circulation. Interestingly, both P2Ns and P2Ns-NAR exhibited a double peak in the plasma persistence properties (FIG. 11). This phenomenon has been previously observed and could be attributed to variable absorption across the intestine or a combination of events such as portal entry and lymphatic drainage.^{11,22,35,36}

TABLE 2
Pharmacokinetic Parameters of
the INS-laden P2Ns and P2Ns-NAR

| PK parameters | AUC (μIU/h/mL) | Cₘₐₓ (μIU/mL) | Tₘₐₓ (h) |
|---|---|---|---|
| P2Ns (INS-laded) | 22920 | 315/301 | 0.25/24 |
| P2Ns-NAR (INS- | 73910 | 757/1432 | 0.5/72 |
| laden | | | |

In vivo insulin kinetics study. Rats were randomly divided into two groups (n=4). Rats received bovine insulin by dispersing insulin-laden P2Ns and P2Ns-NAR nano-systems in water and dosing via oral gavage. All rats received a single dose of insulin of 40 IU of insulin/kg to body weight. Blood samples were withdrawn via tail vein at different time intervals and collected in heparinized tubes up to 96 hours post-dosing. The terminal blood withdrawal (96 hours) was performed via heart puncture. The plasma was separated from the red blood cells by centrifuging the collection tubes at 3000 rpm at 4° C. for 30 minutes. The supernatant/plasma was separated and stored at −80° C. until analysis. All samples were diluted (four times) with calibrator 0 provided in Sigma bovine insulin ELISA kit (as per the instructions in the kit) as a diluent.

Description of experimental results can be found in Heyns I M, et. al., “Rationally Designed Naringenin-Conjugated Polyester Nanoparticles Enable Folate Receptor-Mediated Peroral Delivery of Insulin”, which is incorporated herein in its entirety.

Example: Chemistry and Manufacturing Controls. Naringenin was chemically modified to allow its attachment to carrier material via a chemical linker. The carrier material can be a polyester with single terminal functional carboxyl group or a polyester with multiple functional groups. This conjugation can be extended to any carrier material with a carboxyl group. Two examples are presented below, the first of naringenin conjugated with PLGA and second example of naringenin conjugated with P2s. Respective NMR are shown for each conjugation chemistry. The conjugated groups were laden with several drugs of interest, NAR, UA, insulin, as well as fluorescent particles for imaging studies.

Further studies include ex vivo and in vivo application of the naringenin-conjugated particles. The naringenin-conjugated particles were studied for folate receptor binding and cellular uptake, as shown in the FACS and confocal data examples. In vitro efficacy studies were carried out for LPS, cisplatin, and glucose delivery. In vivo efficacy studies were carried out to determine the efficacy in cisplatin-induced AKI model using NAR-laden and UA-laden naringenin-conjugated particles and well as to determine the enhanced bioavailability of insulin and UA.

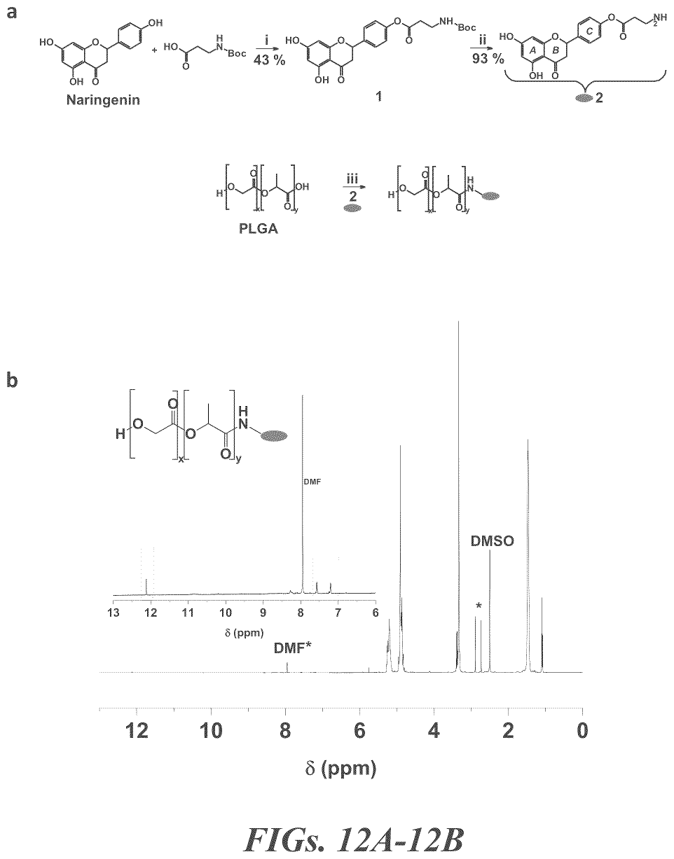

PLGA-NAR particles were synthesized using the route shown in FIG. 12A, which shows steps of i) EDC·HCl, DMAP, dimethylformamide, 0° C. to room temperature; ii) trifluoroacetic acid, methanol, dichloromethane, 0° C.; iii) EDC·HCl, DIEA, dimethylformamide, 0° C. to room temperature. FIG. 12B shows ¹H NMR spectra PLGA-NAR in DMSO-d₆. P2Ns and P2Ns-NAR particles were synthesized using the route shown in FIG. 1A, which shows steps of i) EDC·HCl, DMAP, dimethylformamide, 0° C. to room temperature; ii) trifluoroacetic acid, methanol, dichloromethane, 0° C.; iii) EDC·HCl, DIEA, dimethylformamide, dichloromethane, 0° C. to room temperature. FIG. 1B shows ¹H NMR spectra of 2 and P2s-NAR in DMSO-d₆. Characteristics of the formulated particles are shown in Table 3.

Representative scanning electron micrographs of each insulin-laden and NAR-laden particles are shown in FIGS. 4A-4B, FIGS. 5A-5E, and FIG. 6.

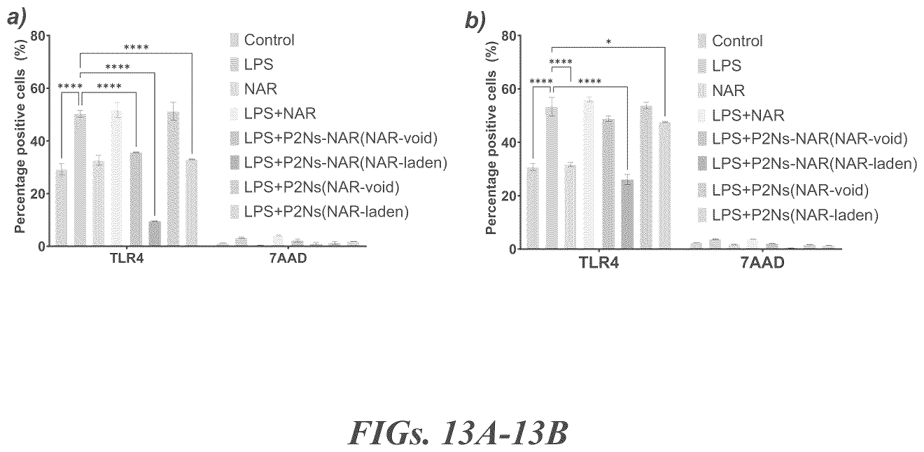

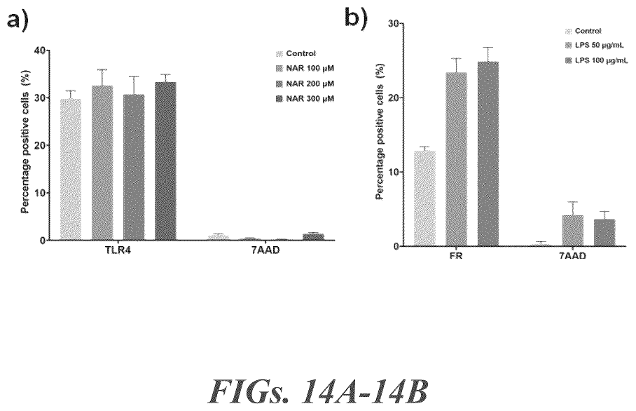

Examples of polymer nanoparticles with an inherent anti-inflammatory action synergistic with encapsulated naringenin. In a first example, LPS-induced inflammation in human intestinal cells, which is indicative of necrotizing enterocolitis (NEC), was studied. FIGS. 13A and 13B show TLR4 expression and cellular death of FHs74 cells upon exposure to 50 g/mL LPS for 1 hour (FIG. 13A) and after 6 hours (FIG. 13B) exposure upon treatment with unfunctionalized nano-formulations versus NAR-decorated nano-formulations. Naringenin-void and naringenin-laden were used in treatment groups (100 μM). Additional examples are shown in FIG. 14A of TLR4 expression and cell viability of FHs74 cells upon 1 hour exposure to varying concentrations of free NAR, and in FIG. 14B, FR expression and cell viability of FHs74 cells upon 1 hour exposure to varying concentrations of LPS.

TABLE 3
Formulation characteristics

| Formulation | Size (nm) | EE (%)* | ZP (mV)** |
|---|---|---|---|
| | Naringenin (Bioactive) | | |
| PLGA-NAR | 230 ± 4 | 40 ± 2 | 12.7 ± 0.9 |
| P2Ns | 259 ± 1 | 52 ± 4 | 20.5 ± 8.3 |
| P2Ns-NAR | 201 ± 3 | 42 ± 8 | −26.4 ± 4.5 |
| | Urolithin A (Bioactive) | | |
| PLGA-NAR | — | — | — |
| P2Ns | 217 ± 15 | 58 ± 1 | 23.6 ± 0.6 |
| P2Ns-NAR | 150 ± 7 | 71 ± 3 | 26.0 ± 4.7 |
| | Insulin (Bioactive) | | |
| PLGA-NAR | — | — | — |
| P2Ns | 289 ± 12 | 77 ± 10 | −16.3 ± 0.1 |
| P2Ns-NAR | 220 ± 22 | 75 ± 12 | 13.9 ± 1.4 |

(*(EE) Encapsulation Efficiency;
**ZP: Zetapotential, at pH = 6-7)

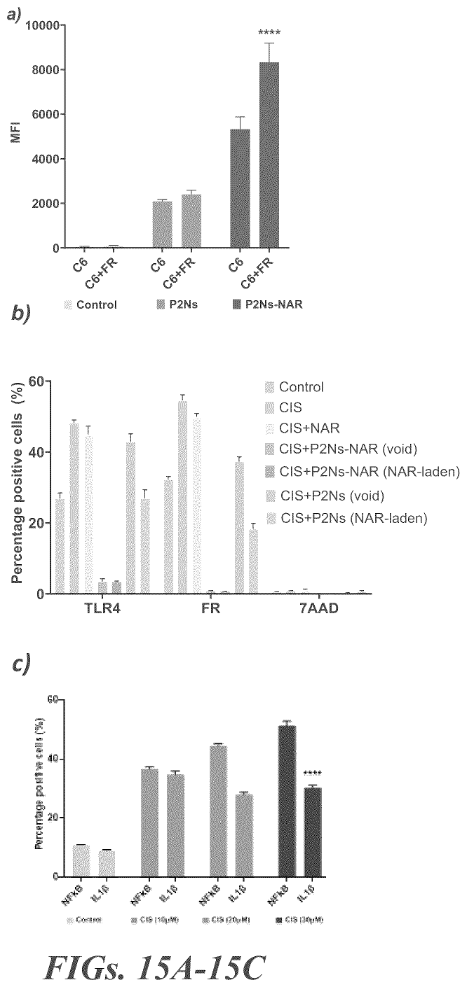

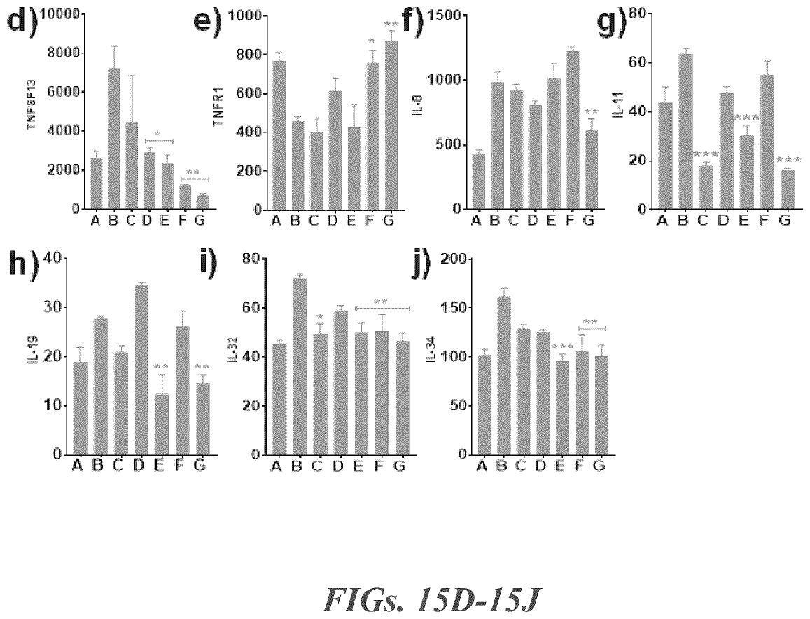

In a third example, Cisplatin-induced inflammation in human kidney cells, which is indicative of acute kidney injury was studied. FIG. 15A shows MFI determined by flow cytometry to evaluate the cellular uptake in HK2 cells. FIG. 15B shows the results of the efficacy study: TLR4, FR expression and cell death of cisplatin treated (20 μM) HK2 cells as a function of treatment groups, determined via FACS. FIG. 15C shows NFkB and IL1β expression of HK2 cells upon exposure to varying concentrations of cisplatin for 6 hours. FIGS. 15D-15J show various inflammation assays for A: control; B: CIS control; C: CIS+Nar; D: CIS+P2Ns (void); G: CIS+P2Ns (NAR-laden); D: CIS+P2Ns+NAR (void); G: CIS+P2Ns+NAR (NAR-laden).

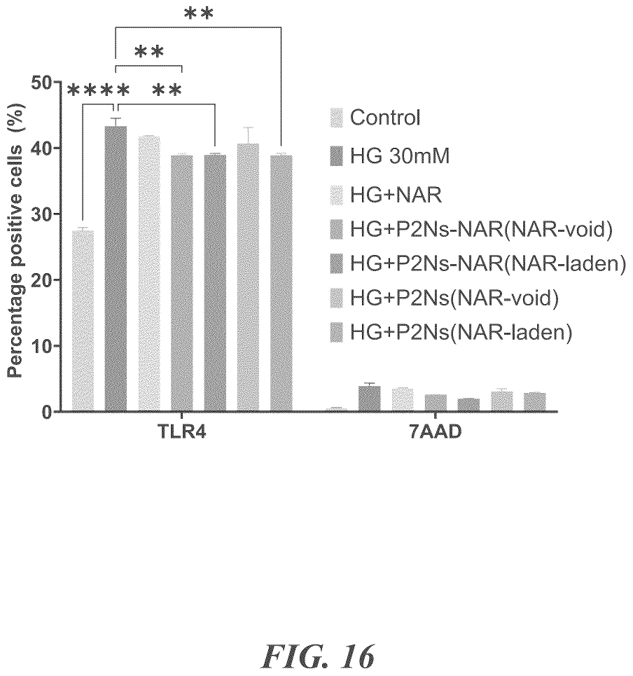

In a second example, FIG. 16 shows TLR4 expression and cellular death of FHs74 cells upon exposure to 30 mM glucose (high glucose) for 3 hours upon treatment with unfunctionalized nano-formulations versus NAR-decorated nano-formulations. Naringenin-void and naringenin-laden were used in treatment groups.

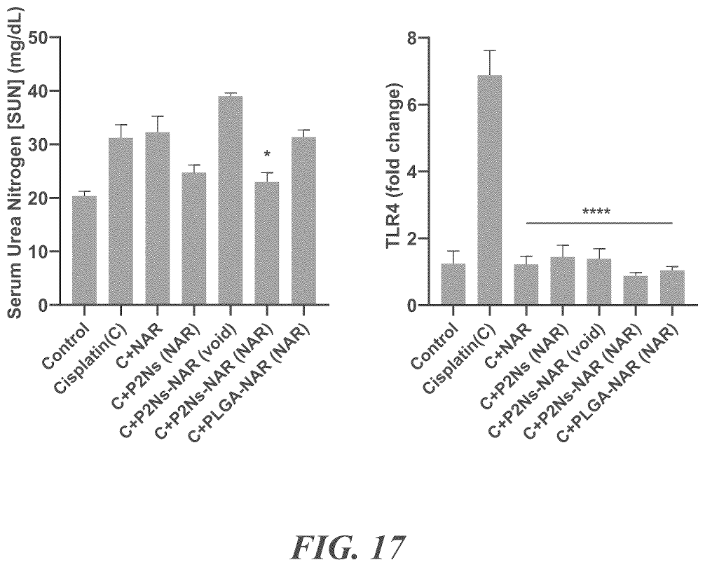

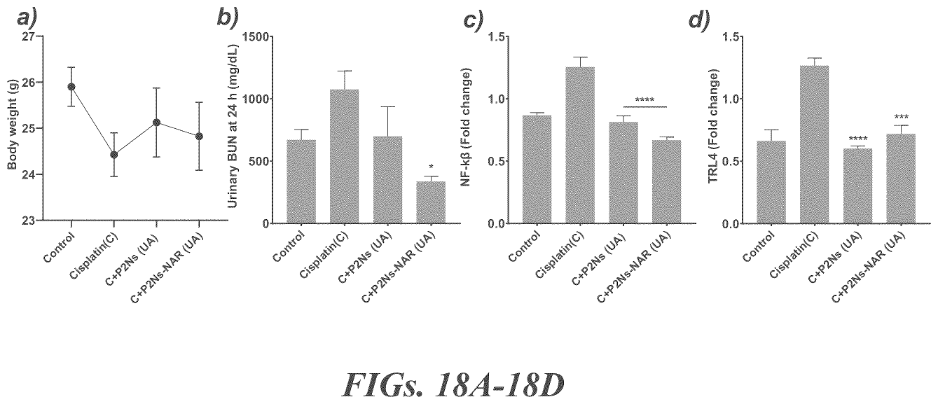

FIG. 17 shows the results of in vivo efficacy studies of NAR, and FIG. 18A-18D show the results of in vivo efficacy studies of UA. Efficacy of oral UA-laden nano-formulations as a function of unfunctionalized nano-formulations versus NAR-decorated nano-formulations in cisplatin-induced acute kidney mice models. P2Ns (UA) dosage was 20 mg/kg and P2Ns-NAR (UA) dosage was 10 mg/kg. FIG. 18A shows body weights of animals at week 4.5; FIG. 18B shows urinary blood urea nitrogen at 24 hours; FIG. 18C shows NF-κβ expression in kidney homogenate; and FIG. 18D shows TLR4 expression in kidney homogenate.

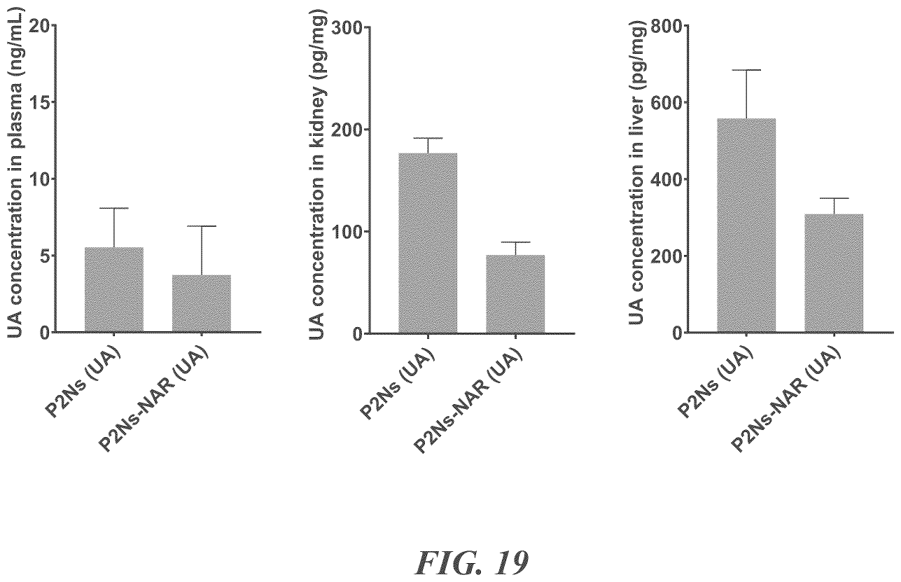

FIG. 19 shows enhanced oral bioavailability of Urolithin A. UA concentration in oral UA-laden nano-formulations as a function of unfunctionalized nano-formulations versus NAR-decorated nano-formulations in cisplatin-induced acute kidney mice models where n=3; determined via LC-MS. P2Ns (UA) dosage was 20 mg/kg and P2Ns-NAR (UA) dosage was 10 mg/kg. The efficacy is shown as UA concentration in plasma, kidney, and liver, respectively.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The expressions “ambient temperature” and “room temperature” as used herein are understood in the art and refer generally to a temperature from about 20° C. to about 35° C.

As used herein, the term “composition” is intended to encompass a product comprising the specified ingredients in the specified amounts, as well as any product which results, directly or indirectly, from a combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example embodiments.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition.

As used herein, the terms “substantially identical reference composition,” “substantially identical reference article,” or “substantially identical reference electrochemical cell” refer to a reference composition, article, or electrochemical cell comprising substantially identical components in the absence of an inventive component. In another exemplary aspect, the term “substantially,” in, for example, the context “substantially identical reference composition,” or “substantially identical reference article,” or “substantially identical reference electrochemical cell” refers to a reference composition, article, or an electrochemical cell comprising substantially identical components and wherein an inventive component is substituted with a common in the art component.

The devices, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods, in addition to those shown and described herein, are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less; however, other combinations of steps, elements, components, and constituents are included, even though not explicitly stated.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove and that many modifications and other embodiments are intended to be included within the scope of the appended claims. Moreover, although specific terms are employed herein, as well as in the claims which follow, they are used only in a generic and descriptive sense and not for the purposes of limiting the described invention nor the claims which follow.

Unless defined otherwise, all technical and scientific terms used herein have the same meanings as commonly understood by one of skill in the art to which the disclosed invention belongs. Publications cited herein and the materials for which they are cited are specifically incorporated by reference.

While aspects can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

### REFERENCES

(1) Xiao, Y.; Tang, Z.; Wang, J.; Liu, C.; Kong, N.; Farokhzad, O. C.; Tao, W. Oral Insulin Delivery Platforms: Strategies to Address the Biological Barriers. Angew. Chem., Int. Ed. 2020, 59 (45), 19787-19795.
(2) Lee, A.; De Mei, C.; Fereira, M.; Marotta, R.; Yoon, H. Y.; Kim, K.; Kwon, I. C.; Decuzzi, P. Dexamethasone-loaded Polymeric Nanoconstructs for Monitoring and Treating Inflammatory Bowel Disease. Theranostics 2017, 7 (15), 3653-3666.
(3) Orellana, E. A.; Abdelaal, A. M.; Rangasamy, L.; Tenneti, S.; Myoung, S.; Low, P. S.; Kasinski, A. L. Enhancing MicroRNA Activity through Increased Endosomal Release Mediated by Nigericin. Mol. Ther. Nucleic. Acids 2019, 16, 505-518.
(4) Roger, E.; Kalscheuer, S.; Kirtane, A.; Guru, B. R.; Grill, A. E.; Whittum-Hudson, J.; Panyam, J. Folic Acid Functionalized Nanoparticles for Enhanced Oral Drug Delivery. Mol. Pharmaceutics 2012, 9 (7), 2103-2110.
(5) Pridgen, E. M.; Alexis, F.; Kuo, T. T.; Levy-Nissenbaum, E.; Karnik, R.; Blumberg, R. S.; Langer, R.; Farokhzad, O. C. Transepithelial Transport of Fc-targeted Nanoparticles by the Neonatal Fc Receptor for Oral Delivery. Sci. Transl. Med. 2013, 5 (213), 213ra167-213ra167.
(6) Du, W.; Fan, Y.; Zheng, N.; He, B.; Yuan, L.; Zhang, H.; Wang, X.; Wang, J.; Zhang, X.; Zhang, Q. Transferrin Receptor Specific Nanocarriers Conjugated with Functional 7 Peptide for Oral Drug Delivery. Biomaterials 2013, 34 (3), 794-806.
(7) Saini, P.; Ganugula, R.; Arora, M.; Kumar, M. The Next Generation Non-competitive Active Polyester Nanosystems for Transferrin Receptor-mediated Peroral Transport Utilizing Gambogic Acid as a Ligand. Sci. Rep. 2016, 6 (1), 1-16.
(8) Saw, P. E.; Song, E.-W. Phage Display Screening of Therapeutic Peptide for Cancer Targeting and Therapy. Protein & Cell 2019, 10 (11), 787-807.
(9) Yan, J.; Gao, T.; Lu, Z.; Yin, J.; Zhang, Y.; Pei, R. Aptamer-targeted Photodynamic Platforms for Tumor Therapy. ACS Appl. Mater. Interfaces 2021, 13 (24), 27749-27773.
(10) Nanna, A. R.; Li, X.; Walseng, E.; Pedzisa, L.; Goydel, R. S.; Hymel, D.; Burke, T. R., Jr.; Roush, W. R.; Rader, C. Harnessing a Catalytic Lysine Residue for the One-step Preparation of Homogeneous Antibody-drug Conjugates. Nat. Commun. 2017, 8 (1), 1112.
(11) Ganugula, R.; Arora, M.; Saini, P.; Guada, M.; Kumar, M. Next Generation Precision-Polyesters Enabling Optimization of Ligand-Receptor Stoichiometry for Modular Drug Delivery. J. Am. Chem. Soc. 2017, 139 (21), 7203-7216.
(12) Ledermann, J.; Canevari, S.; Thigpen, T. Targeting the Folate Receptor: Diagnostic and Therapeutic Approaches to Personalize Cancer Treatments. Ann. Oncol. 2015, 26 (10), 2034-2043.
(13) Zhao, Z.; Ukidve, A.; Kim, J.; Mitragotri, S. Targeting Strategies for Tissue-specific Drug Delivery. Cell 2020, 181 (1), 151-167.
(14) Falciani, C.; Lozzi, L.; Pini, A.; Bracci, L. Bioactive Peptides from Libraries. Chem. Biol. 2005, 12 (4), 417-426.
(15) Schumacher, T. N.; Mayr, L. M.; Minor, D. L., Jr.; Milhollen, M. A.; Burgess, M. W.; Kim, P. S. Identification of D-Peptide Ligands through Mirror-image Phage Display. Science 1996, 271 (5257), 1854-1857.
(16) Devnath, G. P.; Kumaran, S.; Rajiv, R.; Shaha, K. K.; Nagaraj, A. Fatal Folic Acid Toxicity in Humans. J. Forensic Sci. 2017, 62 (6), 1668-1670.
(17) Benavente-Garcia, O.; Castillo, J. Update on Uses and Properties of Citrus Flavonoids: New Findings in Anticancer, Cardiovascular, and Anti-inflammatory Activity. J. Agric. Food Chem. 2008, 56 (15), 6185-6205.
(18) Ji, P.; Yu, T.; Liu, Y.; Jiang, J.; Xu, J.; Zhao, Y.; Hao, Y.; Qiu, Y.; Zhao, W.; Wu, C. Naringenin-loaded Solid Lipid Nanoparticles: Preparation, Controlled Delivery, Cellular Uptake, and Pulmonary Pharmacokinetics. Drug Des. Dev. Ther. 2016, 911-925.
(19) Kirtane, A. R.; Kalscheuer, S. M.; Panyam, J. Exploiting Nanotechnology to Overcome Tumor Drug Resistance: Challenges and Opportunities. Adv. Drug Delivery Rev. 2013, 65 (13-14), 1731-1747.
(20) Albuquerque de Oliveira Mendes, L.; Ponciano, C. S.; Depieri Cataneo, A. H.; Wowk, P. F.; Bordignon, J.; Silva, H.; Vieira de Almeida, M.; Avila, E. P. The Anti-Zika Virus and Anti-tumoral Activity of the Citrus Flavanone Lipophilic Naringenin-based Compounds. Chem. Biol. Interact. 2020, 331, 109218.
(21) Abstiens, K.; Gregoritza, M.; Goepferich, A. M. Ligand Density and Linker Length are Critical Factors for Multivalent Nanoparticle-Receptor Interactions. ACS Appl. Mater. Interfaces 2019, 11 (1), 1311-1320.
(22) Ganugula, R.; Arora, M.; Guada, M.; Saini, P.; Kumar, M. N. R. Noncompetitive Active Transport Exploiting Intestinal Transferrin Receptors for Oral Delivery of Proteins by Tunable Nanoplatform. ACS Macro Lett. 2017, 6 (2), 161-164.
(23) Jain, A.; Jain, S. K. L-Valine Appended PLGA Nanoparticles for Oral Insulin Delivery. Acta Diabetol. 2015, 52, 663-676.
(24) Li, J.; Qiang, H.; Yang, W.; Xu, Y.; Feng, T.; Cai, H.; Wang, S.; Liu, Z.; Zhang, Z.; Zhang, J. Oral Insulin Delivery by Epithelium Microenvironment-adaptive Nanoparticles. J. Controlled Release 2022, 341, 31-43.
(25) Sheng, J.; Han, L.; Qin, J.; Ru, G.; Li, R.; Wu, L.; Cui, D.; Yang, P.; He, Y.; Wang, J. N-trimethyl Chitosan Chloride-coated PLGA Nanoparticles Overcoming Multiple Barriers to Oral Insulin Absorption. ACS Appl. Mater. Interfaces 2015, 7 (28), 15430-15441.
(26) Bhattacharjee, S. DLS and Zeta Potential-What they are and what they are not? J. Controlled Release 2016, 235, 337-351.
(27) Bohn, T. Dietary Factors Affecting Polyphenol Bioavailability. Nutr. Rev. 2014, 72 (7), 429-452.
(28) Yin, X.; Fu, X.; Cheng, H.; Liang, L. α-Tocopherol and Naringenin in Whey Protein Isolate Particles: Partition, Antioxidant Activity, Stability and Bioaccessibility. Food Hydrocoll. 2020, 106, 105895.
(29) Hu, Y.; Chen, D.; Napoleon, J. V.; Srinivasarao, M.; Singhal, S.; Savran, C. A.; Low, P. S. Efficient Capture of Circulating Tumor Cells with Low Molecular Weight Folate Receptor-specific Ligands. Sci. Rep. 2022, 12 (1), 8555.
(30) Low, P. S.; Henne, W. A.; Doorneweerd, D. D. Discovery and Development of Folic-acid-based Receptor Targeting for Imaging and Therapy of Cancer and Inflammatory Diseases. Acc. Chem. Res. 2008, 41 (1), 120-129.
(31) Sawaengsri, H.; Wang, J.; Reginaldo, C.; Steluti, J.; Wu, D.; Meydani, S. N.; Selhub, J.; Paul, L. High Folic Acid Intake Reduces Natural Killer Cell Cytotoxicity in Aged Mice. J. Nutr. Biochem. 2016, 30, 102-107.
(32) Selhub, J.; Rosenberg, I. H. Excessive Folic Acid Intake and Relation to Adverse Health Outcome. Biochimie 2016, 126, 71-78.
(33) Paulos, C. M.; Reddy, J. A.; Leamon, C. P.; Turk, M. J.; Low, P. S. Ligand Binding and Kinetics of Folate Receptor Recycling In Vivo: Impact on Receptor-mediated Drug Delivery. Mol. Pharmacol. 2004, 66 (6), 1406-1414.
(34) Scaglione, F.; Panzavolta, G. Folate, Folic Acid and 5-ethyltetrahydrofolate are not the same thing. Xenobiotica 2014, 44 (5), 480-488.
(35) Amekyeh, H.; Billa, N.; Yuen, K.-H.; Lim, S. C. S. Effect of Food Status on the Gastrointestinal Transit of Amphotericin Bcontaining Solid Lipid Nanoparticles in Rats. AAPS PharmSciTech 2016, 17, 1060-1066.
(36) Chen, C.-H.; Chang, C.-C.; Shih, T.-H.; Aljuffali, I. A.; Yeh, T.-S.; Fang, J.-Y. Self-nanoemulsifying Drug Delivery Systems Ameliorate the Oral Delivery of Silymarin in Rats with Roux-Y Gastric Bypass Surgery. Int. J. Nanomedicine 2015, 10, 2403.
(37) Heyns I M, Ganugula R, Varma T, Allamreddy S, Kumar N, Garg P, Kumar MNVR, Arora M. Rationally Designed Naringenin-Conjugated Polyester Nanoparticles Enable Folate Receptor-Mediated Peroral Delivery of Insulin. ACS Appl Mater Interfaces. 2023 Oct. 4; 15(39):45651-45657.

What is claimed is:

1. A composition comprising a nanoparticle, wherein the nanoparticle comprises a polymer or copolymer conjugated to a naringenin ligand.

2. The composition of claim 1, further comprising a bioactive drug encapsulated by the nanoparticle.

3. The composition of claim 2, wherein the bioactive drug has at least one of limited aqueous solubility or limited tissue penetration.

4. The composition of claim 3, wherein the bioactive drug is naringenin, insulin, GLP-1 agonist, curcumin, urolithin A, cyclosporine A, or combinations thereof.

5. The composition of claim 1, wherein the polymer or copolymer comprises polyethylene glycol, polylactide, poly(lactide-co-glycolide), polycaprolactone, polyglycolide, polyhydroxyalkanoates, polyanhydrides, polyurethanes, polyphosphazenes or combinations thereof.

6. The composition of claim 5, wherein the polymer or copolymer is a block copolymer comprising a polylactide block and a polyethylene glycol block.

7. The composition of claim 6, wherein the block copolymer is a triblock, multiblock, or starblock copolymer.

8. The composition of claim 1, wherein the naringenin ligand is linked to carboxylic end groups of the polymer or copolymer by a linker.

9. The composition of claim 8, wherein the linker comprises an amino acid or bi- and multi-functional diamines, wherein the amino acid, bi- and multi-functional diamines comprise alkyl chains of lengths from C1 to C6.

10. The composition of claim 8, wherein the linker comprises polyethylene glycol (PEG) chains, wherein the PEG chains are less than 5 kDa.

11. The composition of claim 9, wherein the amino acid comprises β-boc alanine, γ-(boc-amino) butyric acid or 6-(boc-amino) caproic acid.

12. The composition of claim 1, wherein a ratio of polymer or copolymer to naringenin ligand is varied from 4 to 12 naringenin ligands to every polymer or copolymer.

13. The composition of claim 1, wherein the nanoparticle is configured to selectively target folate receptors.

14. The composition of claim 1 further comprising one or more detectable compounds chosen from fluorophoric, radio-labeled, and inorganic compounds, wherein the one or more detectable compounds are encapsulated or conjugated to the nanoparticles.

15. A method of producing the composition of claim 2, the method comprising:
synthesizing a polymer or copolymer conjugated with naringenin; and
preparing the nanoparticle by entrapping a desired bioactive drug within the nanoparticle.

16. The composition of claim 2, further comprising a suspension of freeze-dried nanoparticles in water or milk, thereby forming uniform colloidal system.

17. A method of administration, the method comprising administering the composition of claim 16 to a subject.

18. The method of administration of claim 17, wherein the administering is carried out orally or perorally.

19. The method of administration of claim 17, wherein the administering is by nose, skin, or injection.

20. The method of administration of claim 17, wherein the composition is administered in response to short-term or chronic inflammation of a subject.
